(19) United States
(12) Patent Application Publication
(10) Pub. No.: US 2008/0243078 A1
(43) Pub. Date: Oct. 2, 2008

(54) SINGLE CONTROL BUTTON TYPE AUTOMATIC INFUSION PUMP OPERATION METHOD

(75) Inventor: Yuan-Yung Lee, Taipei City (TW)

Correspondence Address: PACIFIC HOSPITAL SUPPLY CO., LTD., P.O. BOX 108-00403, TAIPEI 106, TW

(73) Assignee: PACIFIC HOSPTIAL SUPPLY CO., LTD., Taipei City (TW)

(21) Appl. No.: 11/695,068

(22) Filed: Apr. 2, 2007

(51) Int. Cl. A61M 1/00

(52) U.S. Cl. 604/151

(57) ABSTRACT

A single control button type automatic infusion pump comprised of a circuit board, a motor pump, a three-way connector, a buzzer and a power adapter is disclosed. The circuit board has a controller to control the operation of the motor pump and the buzzer and a sensor to detect the pressure or status of the infusion bag/nutrition bag and the three-way connector. The three-way connector is a connection interface for enabling the motor pump to increase or reduce the pressure of the external infusion bag or nutrition bag. By means of a single button design, the single control button type automatic infusion pump automatically controls the pressure of the infusion bag/nutrition bag and detects its operation.

### BACKGROUND OF THE INVENTION

1. Field of the Invention

The present invention relates to medical implements and more particularly, to a single control button type automatic infusion pump, which is comprised of a circuit board, a motor pump, a three-way connector, a buzzer and a power adapter, and which is operated by means of the control of a single control button.

2. Description of the Related Art

A typical medical system uses a computer network to control the operation of medical implements. The use of a computer-controlled medical system greatly improves efficiency. However, it requires a skilled person to operate the system.

Further, during a surgical operation, the nurse may need to introduce a saline, blood or other solution from an infusion bag, blood bag or nutrition bag into the vein of the patient. During introduction of a saline, blood or other solution, the nurse may have to check the infusion status of the infusion bag, blood bag or nutrition bag frequently. If the pressure of the infusion bag, blood bag or nutrition bag is insufficient, the nurse may have to squeeze the infusion bag, blood bag or nutrition bag with the hands to increase the pressure, enabling the saline, blood or other solution to be introduced into the patient vein at a proper speed.

There are many automatic infusion pumps developed for giving pressure to an infusion bag, blood bag or nutrition bag automatically. However, these automatic infusion pumps are still not satisfactory in function because of the following drawbacks:

1. These conventional automatic infusion pumps have a number of buttons for different pressure controls. Before using an automatic infusion pump, the nurse or medical personnel must memorize the arrangement of the automatic infusion pump or read the instructions of the automatic infusion pump carefully to prevent an error. Therefore, the use of these conventional automatic infusion pumps requires a complicated learning procedure.

2. These conventional automatic infusion pumps require much installation space. The multi-button design complicates the molding of the apparatus, resulting in a high manufacturing cost.

3. The complicated multi-button design gives a stress to the patient, increasing patient agitation.

### SUMMARY OF THE INVENTION

The present invention has been accomplished under the circumstances in view.

It is one object of the present invention to provide a single control button type automatic infusion pump, which uses one single button for operation control so that a nurse or medical-care personnel needs not to memorize the arrangement of the single control button type automatic infusion pump and can use the single control button type automatic infusion pump to introduce a saline or other solution from an infusion bag or nutrition bag into the vein of a patient after a simple learning procedure.

It is another object of the present invention to provide a single control button type automatic infusion pump, which uses only one button for operation control and, which is a small-sized design and has a low molding cost.

It is still another object of the present invention to provide a single control button type automatic infusion pump, which has a simple structure that reduces patient agitation.

To achieve these and other objects of the present invention, the single control button type automatic infusion pump is comprised of a circuit board, a motor pump, a three-way connector, a buzzer and a power adapter. The circuit board comprises a controller that controls the operation of the motor pump and the buzzer, and a sensor that detects the pressure of the infusion bag/nutrition bag and the status of the three-way connector. The three-way connector is a connection interface for enabling the motor pump to increase or reduce the pressure of the external infusion bag or nutrition bag. The buzzer is adapted to produce a warning sound signal. The power adapter is a power supply module adapted to provide the necessary working voltage to all electric/electronic parts of the single control button type automatic infusion pump.

### BRIEF DESCRIPTION OF THE DRAWINGS

FIG. 1 is a sectional view of a single control button type automatic infusion pump in accordance with the present invention.

FIG. 2 is a system block diagram of the single control button type automatic infusion pump in accordance with the present invention.

FIG. 3 is an operation flow of the single control button type automatic infusion pump in accordance with the present invention.

FIG. 4 is a detailed block diagram showing the operation of the single control button type automatic infusion pump in accordance with the present invention.

### DETAILED DESCRIPTION OF THE PREFERRED EMBODIMENT

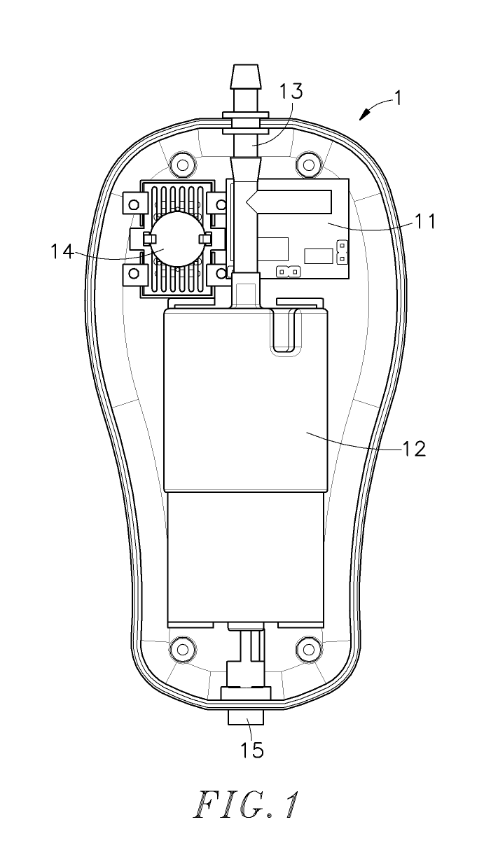

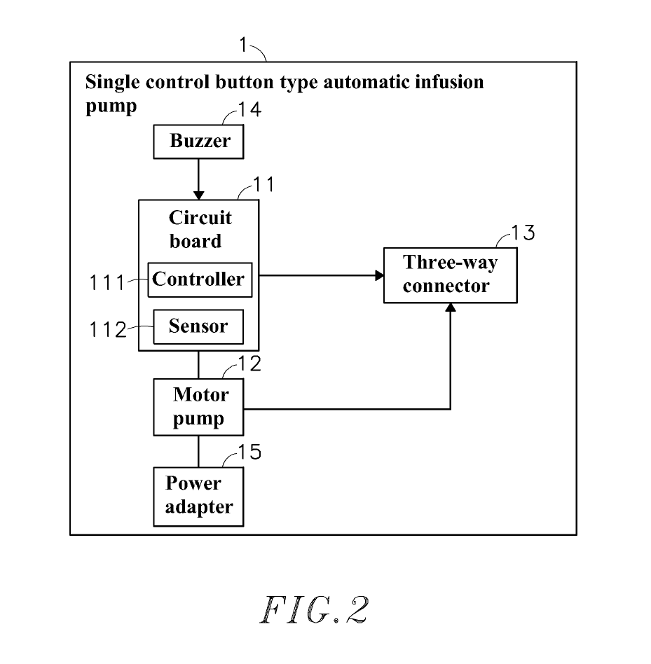

Referring to FIGS. 1 and 2, a single control button type automatic infusion pump 1 in accordance with the present invention is shown comprising a circuit board 11, a motor pump 12, a three-way connector 13, a buzzer 14 and a power adapter 15. The circuit board 11 controls the pumping operation of the single control button type automatic infusion pump 1. The circuit board 11 comprises a controller 111 that controls the operation of the motor pump 12 and the buzzer 14, and a sensor 112 that detects the pressure or status of the infusion bag or nutrition bag (not shown) and the three-way connector 13. The three-way connector 13 is a connection interface for enabling the motor pump 12 to increase or reduce the pressure of the external infusion bag or nutrition bag. The buzzer 14 is adapted to produce a warning sound signal. The power adapter 15 is a power supply module adapted to provide the necessary working voltage to all electric/electronic parts of the single control button type automatic infusion pump 1.

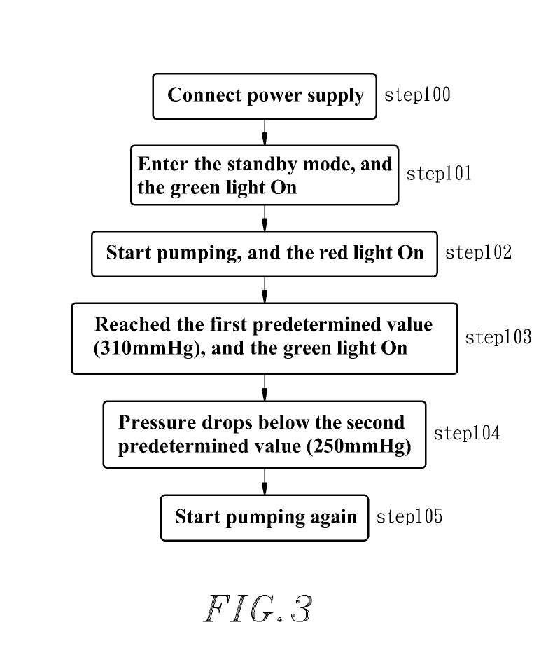

FIG. 3 is an operation flow of the present invention. When the single control button type automatic infusion pump 1 is used to control infusion of an infusion bag or nutrition bag into the patient's vein, is proceeds as follows:

(100) Connect power supply;
(101) A green light is turned on to enter a standby mode;
(102) Start pumping and turn on a red light;
(103) Turn on the green light when the pressure reached a first predetermined value (310 mmHg);
(104) The pressure drops below a second predetermined value (250 mmHg);
(105) Start pumping again.

In general, when using the single control button type automatic infusion pump 1, the first step is to connect power supply to the single control button type automatic infusion pump 1. Upon connection of power supply to the single control button type automatic infusion pump 1, a green light is turned on to indicate a standby mode. When a user pressed a button to start the single control button type automatic infusion pump 1, the motor pump 12 is started to pump, giving a pressure to the infusion bag or nutrition bag. When the sensor 112 detected that the pressure of the infusion bag or nutrient bag has dropped below the predetermined low value after a certain period of time during working of the single control button type automatic infusion pump 1, the controller 111 of the circuit board 11 starts the motor pump 12 to increase the pressure of the infusion bag or nutrient bag to the level above the first predetermined value.

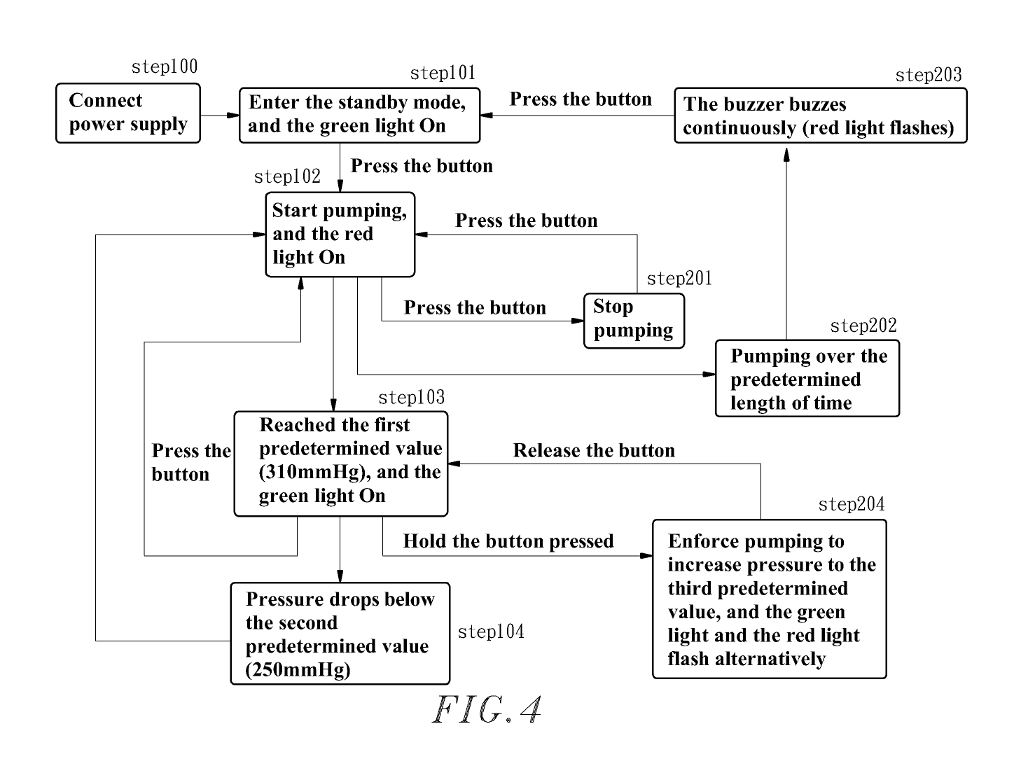

The operation of the present invention includes other sub steps. As shown in FIG. 4, if the user presses the button again during step 102, the single control button type automatic infusion pump 1 will be switched off, and the green light will be turned (step 201). If the user presses the button again during step 201, it will proceed to step 102 to start pump and to turn on the red light. If the pumping action during step 102 surpasses a predetermined period (for example 70 seconds), the single control button type automatic infusion pump 1 will automatically detect the improper installation or damage status of the three-way connector 13 and will drive the buzzer 14 to produce a continuous warning sound signal (step 203), and the single control button type automatic infusion pump 1 will return to step 101 if the user presses the button again at this time. Further, if the user presses the button and holds the button in the pressed status during step 103, the single control button type automatic infusion pump 1 will force the motor pump 12 to increase the pressure of the infusion bag or nutrition bag to a third predetermined value (step 204), and the green light and the red light of the single control button type automatic infusion pump 1 will flash alternatively. If the user releases the button during this enforced pumping action, the single control button type automatic infusion pump 1 will maintain the pressure at the level that the user released the hand from the button.

In order to eliminate human error in the aforesaid pumping process, the single control button type automatic infusion pump 1 can be set to provide an upper limit pressure value, i.e., the aforesaid third predetermined value. If the user presses the button again during pumping, the controller 111 will stop the pumping action to prevent an excessive high pressure of the infusion bag or nutrition bag. Further, in the aforesaid embodiment, the green light is to indicate the standby mode and the red light is to indicate the pumping action. Alternatively, the green light can be used to indicate normal functioning, and the red light can be used to indicate an abnormal condition.

As stated above, the invention provides a single control button type automatic infusion pump 1, which has the following features:

1. The single control button type automatic infusion pumps 1 uses one single button for operation control. Therefore, a nurse or medical-care personnel needs not to memorize the arrangement of the single control button type automatic infusion pump 1. Through a simple learning procedure, any person can use the single control button type automatic infusion pump 1 to introduce a saline or other solution from an infusion bag or nutrition bag into the vein of a patient.

2. The single control button type automatic infusion pump 1 uses only one button for operation control. The molding cost for the single control button type automatic infusion pump 1 is low. Further, the single control button type automatic infusion pump 1 is a small-sized design that does not require much installation space.

3. If the pumping action surpasses a predetermined length of time, the single control button type automatic infusion pump 1 will automatically check installation or damage of the three-way connector 13, and will further drives the buzzer 14 to output a continuous warning sound, assuring safety use of the single control button type automatic infusion pump 1.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A single control button type automatic infusion pump operation method comprising the steps of:
(a) connecting power supply;
(b) entering a standby mode and turning on a first indication light;
(c) pressing a control button to start pumping and to turn on a second indication light;
(d) stopping the pumping action and turning off said second indication light and turning on said first indication light when the pressure reached a first predetermined value;
(e) starting the pumping action again when the pressure dropped below a second predetermined value.

2. The single control button type automatic infusion pump operation method as claimed in claim 1, wherein when the user presses the control button of the single control button type automatic infusion pump during step (c), the pumping action is off.

3. The single control button type automatic infusion pump operation method as claimed in claim 2, wherein when the user presses the control button of the single control button type automatic infusion pump again after stoppage of the pumping action, the pumping action is started again.

4. The single control button type automatic infusion pump operation method as claimed in claim 1, further comprising a sub step of driving a buzzer to produce a warning sound and a warning light to produce a visual warning signal when the pumping action continues and surpasses a predetermined length of time during step (c).

5. The single control button type automatic infusion pump operation method as claimed in claim 4, when the user presses the control button of the single control button type automatic infusion pump during the operation of said buzzer and said warning light, said buzzer and said warning light are turned off, and the single control button type automatic infusion pump is returned to said standby mode.

6. The single control button type automatic infusion pump operation method as claimed in claim 1, wherein when the user presses the control button of the single control button type automatic infusion pump and keeps the control button pressed during the pumping action in step (c), the pumping action is continued to increase the pressure over said first predetermined value to a third predetermined value, which is a set upper limit pressure value of the single control button type automatic infusion pump.

7. The single control button type automatic infusion pump operation method as claimed in claim 6, wherein said first indication light and said second indication light are driven to flash alternatively during the pumping action to increase the pressure over said first predetermined value to said third predetermined value.
